(19) United States
(12) Patent Application Publication
(10) Pub. No.: US 2021/0187201 A1
(43) Pub. Date: Jun. 24, 2021

(54) INJECTION DEVICE WITH A PRESELECTOR

(71) Applicant: Sanofi-Aventis Deutschland GMBH, Frankfurt am Main (DE)

(72) Inventors: Michael Helmer, Frankfurt am Main (DE); Michael Jugl, Frankfurt am Main (DE); Michael Schabbach, Frankfurt am Main (DE); Matthias Rau, Rüsselsheim (DE); Julian Kersting, Rüsselsheim (DE); Andrea Thurnes, Rüsselsheim (DE)

(21) Appl. No.: 16/761,295

(22) PCT Filed: Nov. 5, 2018
(86) PCT No.: PCT/EP2018/080077

§ 371 Date: May 4, 2020

(30) Foreign Application Priority Data
Nov. 7, 2017 (EP) 17200313.9

(51) Int. Cl. A61M 5/315

(52) U.S. Cl. CPC A61M 5/31536; A61M 2005/3154; A61M 5/31551

(57) ABSTRACT

An injection device includes an elongated housing extending along a longitudinal axis and having a distal end and a proximal end, a dose tracker being at least one of translationally or rotationally displaceable relative to the housing and being displaceable between a zero dose positional state and a maximum dose positional state relative to the housing, the positional states indicating a dose, wherein one of the elongated housing and the dose tracker includes at least one tracking stop feature, and a preselector displaceable relative to the housing between at least two preselection positional states thereby defining the maximum dose positional state of the dose tracker, wherein the preselector includes at least one preselector stop feature configured to mechanically engage with the at least one tracking stop feature to block a displacement of the dose tracker beyond the maximum dose positional state.

### CROSS REFERENCE TO RELATED APPLICATIONS

The present application is the national stage entry of International Patent Application No. PCT/EP2018/080077, filed on Nov. 5, 2018, and claims priority to Application No. EP 17200313.9, filed on Nov. 7, 2017, the disclosures of which are incorporated herein by reference.

### TECHNICAL FIELD

The present disclosure relates in one aspect to an injection device, such as a pen-type injector for setting and dispensing of a dose of a medicament. In particular, the disclosure relates to an injection device comprising a preselector configured to limit a maximum dose that can be set and dispensed by the injection device.

### BACKGROUND

Injection devices for setting and dispensing a single or multiple doses of a liquid medicament are known. Generally, such devices have substantially a similar purpose as that of an ordinary syringe.

Injection devices, in particular pen-type injectors have to meet a number of user-specific requirements. For instance, with patient's suffering chronic diseases, such as diabetes, the patient may be physically infirm and may also have impaired vision. Suitable injection devices especially intended for home medication therefore need to be robust in construction and should be easy to use. Furthermore, manipulation and general handling of the device and its components should be intelligible and easy understandable. Moreover, the dose setting as well as dose dispensing procedure must be easy to operate and has to be unambiguous.

Typically, such devices comprise a housing including a particular cartridge holder, adapted to receive a cartridge at least partially filled with the medicament to be dispensed. Such devices further comprise a drive mechanism, usually having a displaceable piston rod which is adapted to operably engage with a piston of the cartridge. By means of the drive mechanism and its piston rod, the piston of the cartridge is displaceable in a distal direction or dispensing direction and may therefore expel a predefined amount of the medicament via a piercing assembly, which is to be releasably coupled with a distal end section of the housing of the injection device.

The medicament to be dispensed by the injection device is provided and contained in a multi-dose cartridge. Such cartridges typically comprise a vitreous barrel sealed in a distal direction by means of a pierceable seal and being further sealed in proximal direction by the piston. With reusable injection devices an empty cartridge is replaceable by a new one. In contrast, injection devices of disposable type are to be discarded when the medicament in the cartridge has been dispensed or used-up.

### SUMMARY

The present disclosure provides an injection device with increased patient safety and comprises a mechanism that prevents unintended overdosing of a medicament. The injection device provides a limited capability to set and to dispense doses of different sizes. The injection device at least temporally provides setting and dispensing of only one or a few differently sized doses. In particular, the injection device is configured to allow and to enable repeated and multiple setting and dispensing of only a few, e.g. of two, three or four differently sized doses of the medicament.

The present disclosure further provides an injection device being intuitive and simple to use even for patients suffering side effects or having an impaired vision. The injection device provides a clearly visible feedback and/or mechanical or haptic feedback to a user thereby indicating that a dose of a predefined size is set and that the device is ready for starting a dispensing procedure.

Advantageously, a maximum size of a dose that can be dispensed or expelled from the cartridge can be limited to prevent unintended overdosing of the medicament.

In one aspect there is provided an injection device for setting and for injecting a dose of a medicament. The injection device comprises an elongated housing extending along a longitudinal axis and having a distal end and a proximal end. The distal end is closest to a dispensing end of the housing whereas the proximal end is located at an opposite end of the elongated housing. Typically and in use, the proximal end is provided with at least one actuator, such as a dose dial, a preselector and/or a trigger in order to set a dose and to trigger dispensing of the dose.

The injection device further comprises a dose tracker being at least one of translationally or rotationally displaceable relative to the housing. The dose tracker is displaceable between a zero dose positional state and a maximum dose positional state relative to the housing for setting of the dose. The positional state of the dose tracker relative to the housing is indicative of a size of the dose. In the present context a positional state includes a position of the dose tracker relative to the housing as well as an orientation of the dose tracker relative to the housing.

At least one of the elongated housing and the dose tracker comprises at least one tracking stop feature. The injection device further comprises a preselector displaceable relative to the housing between at least two preselection positional states thereby defining the maximum dose positional state of the dose tracker. The preselector comprises at least one preselector stop feature. The preselector stop feature is configured to mechanically engage with the at least one tracking stop feature in order to block and to impede a displacement of the dose tracker beyond the maximum dose positional state.

The preselector defines a maximum length of a displacement path for the dose tracker relative to the housing. The length of the displacement path correlates to the size of the dose actually set and to be dispensed during a subsequent dispensing operation of the injection device. During setting of a dose the preselector is stationary relative to the housing. It may be fixed or locked to the housing. During setting of the dose and for setting of the dose the dose tracker is displaceable relative to the housing. At the end of the dose setting procedure the dose tracker is in the maximum dose positional state that is defined by the preselection positional state of the preselector. Once the dose tracker has reached the maximum dose positional state a dispensing operation for expelling a dose of the medicament may commence or may be triggered.

The process of dose setting while the preselector is in a predefined preselection positional state is either conducted by the user himself or is conducted automatically. The dose tracker is displaceable from the zero dose positional state to the maximum dose positional state either under the action of a mechanical drive, such as a spring or the dose tracker is manually displaced by a user interacting with an actuator, such as a trigger or a dose dial. For dispensing of a dose the user may exert a dispensing force onto a trigger of the injection device. During dose dispensing the dose tracker returns from the maximum dose positional state to the zero dose positional state. During dispensing of a dose the dose tracker may be displaced against the action of the mechanical drive, hence against the action of the spring.

The interaction between the preselector and the dose tracker is beneficial for that the user does not have to take care about a setting of a correct dose. The preselector is particularly applicable with injection devices generally providing numerous different positional states for the dose tracker starting from which a dose injection procedure may commence. With the preselector the capability of the injection device to set and to dispense differently sized doses of the medicament is limited to only one dose size at a time. It is intended that the dose tracker is always displaced from the zero dose positional state to the maximum dose positional state. The mechanical interaction between the preselector stop feature and the tracking stop feature automatically limits and prevents an overdosing and hence a displacement of the dose tracker beyond the maximum dose positional state.

In effect, setting of a dose is provided through the interaction of the preselector and the dose tracker. The preselector defines a maximum dose without actually setting the dose whereas the dose tracker is to be displaced relative to the housing for setting of a dose without selecting or defining the size of the dose. Dose size selection is exclusively performed and conducted by the preselector. Setting of a dose of predefined size governed by the configuration of the preselector is exclusively performed and conducted by some other device component, such as a dose dial.

The total displacement of the dose tracker is defined by the preselection positional state of the preselector. Once the preselector is correctly positioned in a predefined preselection positional state the end user does no longer have to take care about the setting of a correct dose. This is of particular benefit in a scenario where the preselector is for instance manipulated or is exclusively configurable by authorized persons, such as caregivers. In this way a patient or user of the injection device is hindered from modifying the preselection positional state of the preselector. Movement or configuration of the preselector may require a certain tool or may require a partial disassembly of the injection device. For instance, the preselector may be covered by a cover, such as an adhesive label

In this way, the caregiver may be provided with the exclusive possibility to limit and to restrict a maximum size of a dose that can be set and dispensed by the injection device.

In examples wherein the at least one tracking stop feature is provided on the elongated housing the preselector is at least translationally fixed to the dose tracker. In particular, the preselector may be locked to the dose tracker with regard to the longitudinal direction. A longitudinal displacement or a translational displacement of the dose tracker relative to the elongated housing is then equally transferred to the preselector and vice versa. In this way the preselector moves in unison with the dose tracker. When reaching the maximum dose positional state the preselector stop feature engages with the tracking stop feature of the housing. Here, for selecting at least one of the two preselection positional states the preselector may be rotatable relative to the dose tracker for selection of the preselection positional state among a plurality of available preselection positional states.

In other examples, wherein the tracking stop feature is provided on the dose tracker the preselector may be translationally fixed relative to the housing. The preselector may be rotationally supported on the housing or the preselector may be rotatable or slidable along a tangential or circumferential direction of the housing. The housing may comprise a substantially tubular or cylindrical shape. Typically, the preselector is fixed to the housing with regard to the longitudinal axis. A longitudinal displacement of the dose tracker from the zero dose positional state to the maximum dose positional state then brings the tracking stop feature in engagement or in abutment with the preselector stop feature. In this way a further displacement of the dose tracker along the longitudinal axis of the injection device is effectively blocked and impeded.

Use of the injection device by a patient becomes safer since the injection device is preconfigured for only one predefined dose size. By means of the preselector the injection device originally configured as a variable dose size injection device can be transferred or transformed into a fixed dose injection device preconfigured to set and to dispense numerous doses of a medicament of a predefined size.

The preselector stop feature and the tracking stop feature may comprise mutually corresponding stop faces, e.g. extending in circumferential and/or radial direction so as to engage axially. Alternatively or additionally the preselector stop feature and the tracking stop feature may comprise mutually corresponding stop faces extending in axial direction and radial direction so as to engage circumferentially. When configured to engage axially, the mutual engagement of the preselector stop feature and the tracking stop feature provides an axial stop thereby impeding and blocking a longitudinal or axial translation of the dose tracker beyond the maximum axial dose positional state.

In another example at least a proximal end of the dose tracker protrudes proximally from a proximal end of the housing when in the maximum dose positional state. A longitudinal distance between the zero dose positional state and the maximum dose positional state correlates with the size of the dose. In the zero dose positional state a proximal end of the dose tracker may be located distally from a proximal end of the housing. Alternatively, the proximal end of the dose tracker may align with a proximal end of the housing. When in the maximum dose positional state and when protruding proximally from a proximal end of the housing the dose tracker or an actuator, such as a trigger operatively connected to the dose tracker, may be depressible in distal direction in order to trigger, to commence and/or to control a dose dispensing action of the injection device.

The dose tracker protruding proximally from a proximal end of the housing provides a rather intuitive, at least a visible or haptically discernible indication to a user, that the maximum dose positional state has been reached and that the injection device is prepared and ready for conducting a dose dispensing operation.

In another example the preselector is lockable relative to the housing in any of the at least two preselection positional states. The preselector may be lockable relative to the housing by means of a first locking feature provided on the preselector and by means of a second locking feature provided on the housing. For instance, the first locking feature may comprise a detent structure and the second locking feature may comprise a counter detent structure. One of the detent structure and the counter detent structure comprises a protrusion whereas the other one of the detent structure at the counter detent structure comprises numerous recesses to receive and to positionally lock the protrusion. The recesses are spaced from each other with regards to a displacement direction of the preselector between the at least two preselection positional states. The recesses may be spaced equidistantly.

Typically, the preselector is accessible to a user from outside the housing. The preselector may flush with an outside surface of the housing. Alternatively, the preselector may protrude from an outside surface of the housing or the preselector may be arranged in a recess of the outside surface of the housing. Eventually and for impeding unauthorized access to the preselector the preselector may be covered by a protector, such as an adhesive label or the like cover.

In one example the injection device further comprises a spring to urge the dose tracker in the proximal direction relative to the housing. In this way an automated dose setting can be provided. By means of the spring the dose tracker can be automatically displaced from an initial position towards and into the at least a first activation position. In a further example the injection device comprises an interlock to lock the dose tracker in the initial position relative to the housing. By means of the interlock the dose tracker can be immobilized relative to the housing at least with regard to the longitudinal or axial direction. It can be fixed to the housing by means of the interlock to prevent unintended dose setting and/or dose dispensing.

In a further example the injection device comprises a release member connected to one of the housing and the dose tracker. The release member is selectively engageable to the other one of the housing and the dose tracker in order to lock the dose tracker to the housing when in the zero dose positional state. The release member may be operable engaged or may be operable engageable with the interlock. The release member may be a component of the interlock. The release member may comprise a trigger or an actuator that can be actuated, i.e. depressed or dialed by a user in order to initiate an automated dose setting procedure. By means of the mutual interaction of the spring, the interlock and the at least one release member the process of dose setting can be facilitated. For setting of a dose a user only has to actuate or to depress the at least one release member so as to release the interlock. With a released interlock the dose tracker is released with regard to a longitudinal displacement. It is then free to be moved under the action of the relaxing spring.

The release member may comprise a first locking feature while the housing or the dose tracker may comprise a correspondingly shaped second locking feature to engage with the first locking feature of the release member. The first and second locking features may comprise or may form a positive engagement between the housing and the dose tracker. The release member may be directly attached or connected to one of the housing of the dose tracker. The release member may be also indirectly attached or connected to one of the housing and the dose tracker. The release member may be connected or integrally formed with a further component of the injection device that is operably engaged with one of the release member and the housing.

For releasing the dose tracker from the housing the release member is one of rotationally or longitudinally displaceable relative to the housing. The release member may be rotatable or pivotable or depressible relative to the housing with regard to the longitudinal axis, with regard to a radial direction or with regard to a tangential direction of the tubular shaped housing.

The release member is of particular benefit for such examples, wherein the dose tracker is automatically displaceable from the zero dose positional states to the maximum dose positional state. Here, the injection device may comprise a mechanical drive operable to displace the dose tracker from the zero dose positional state to the maximum dose positional state. Once released by the release member the dose tracker may travel or may be displaced automatically from the zero dose positional state to the maximum dose positional state under the effect of the mechanical drive. In this way a rather automated dose setting can be provided which is rather user-friendly and failure safe.

According to another example the release member comprises an annular ring rotationally supported at the proximal end of the housing. One of an inside surface of the annular ring and an outside surface of the dose tracker comprises at least one catch to engage with a protrusion of the other one of the inside surface of the annular ring or the outside surface of the dose tracker. For releasing the dose tracker from the housing the annular ring is intended to be rotated along a tangential or circumferential direction of the housing. In this way the at least one catch disengages from the at least one protrusion thereby liberating a displacement of the dose tracker relative to the housing.

In typical examples, the dose tracker is translationally supported relative to the housing. When released the dose tracker or a component connected therewith is slidably and/or rotationally displaceable from the zero dose positional state to the maximum dose positional state.

In another example the injection device comprises a spring having a first end operably connected to the housing and having a second end operably connected to the dose tracker for displacing the dose tracker from the zero dose positional state to the maximum dose positional state. The spring may serve and provide a mechanical drive for automatically displacing the dose tracker at least from the zero dose positional state to the maximum dose positional state. In this way the spring provides an automated dose setting as soon as a movement of the dose tracker relative to the housing is allowed or released by actuation of the release member.

The spring is implemented in an injection device. It provides a long-lasting, durable and failure safe mechanical drive to apply a driving force to the dose tracker during a dose setting procedure. During a dose dispensing procedure the dose tracker returns from the maximum dose positional state to the zero dose positional state. This displacement is typically conducted manually by a force exerted and provided by a user of the device. The displacement of the dose tracker from the maximum dose positional state to the zero dose positional state is to be conducted against the action of the spring.

In this way mechanical energy exerted to and provided to the dose tracker during dispensing of a dose is at least partially stored in the spring. For a subsequent dose setting procedure this mechanical energy may be release again. Insofar the injection device is configured for repeated use and hence for setting and dispensing of a multitude of doses of the medicament.

The first end of the spring may be directly connected to the housing or may be indirectly connected to the housing. The first end may be connected to a further component of the injection device, which further component is at least one of rotationally or translationally locked to the housing. In the same way also the second end of the spring may either be directly connected to the dose tracker or it may be connected to a component of the injection device that is at least one of translationally or rotationally locked to the dose tracker.

According to another example the spring comprises a cylindrically shaped torsion spring. The spring may enclose at least a portion of the dose tracker. Alternatively, the spring is arranged inside a hollow portion of the dose tracker. The torsion spring is configured to induce a torque to the dose tracker relative to the housing. A torsion spring is of particular benefit where the dose tracker is rotationally supported on the housing or wherein the dose tracker is threadedly engaged with the housing.

The first end of the torsion spring may be directly connected to the dose tracker while the second end of the torsion spring may be directly connected to the housing of the injection device. The first end of the torsion spring may be also connected to a further device component in a torque proof engagement with the dose tracker. Also, a second end of the torsion spring may be directly connected to a further device component in a torque proof engagement with the housing of the injection device. This provides an increased flexibility to integrate the torsion spring inside the housing and to integrate the torsion spring in a dose setting mechanism of the injection device.

According to a further example the dose tracker comprises a tracking sleeve that is threadedly engaged with the housing. The tracking sleeve may be cylindrically shaped. The tracking sleeve may be located inside the housing. When threadedly engaged with the housing the first end of the spring may be directly connected to the dose tracker whereas the second end of the spring may be directly connected to the housing. In this way and when released by actuation of the release member the dose tracker is free to rotate or to wound helically relative to the housing under the action of the spring.

In this way a fully automated dose setting procedure can be provided by the injection device. The end user does no longer have to take care about the dose setting process. He may only actuate the release member for that the dose setting mechanism automatically displaces the dose tracker into the maximum dose positional state. Selection or modification of the dose to be set is exclusively conducted by the preselector. The preselector remains fixed and stationary relative to the housing during dose setting as well as during dose dispensing. For setting and dispensing numerous doses of equal size the preselector may remain stationary relative to the housing. At least the preselector remains stationary with regard to a displacement direction along which the preselector has to be displaced for bringing the preselector from one preselection positional state to another preselection positional state. User interaction with the injection device may be limited to an actuation of the release member for setting of the dose and to the application of a driving force to a trigger or the like actuator of the injection device in order to trigger or to control a dose dispensing procedure.

In another example the tracking stop feature comprises a radial protrusion protruding from a sidewall of the dose tracker. Typically, the radial protrusion may protrude from an outside facing surface of the tracking sleeve. The radial protrusion of the tracking stop feature may comprise a radially outwardly extending protrusion when the tracking stop feature is provided on the dose tracker. When provided on the housing the radial protrusion of the tracking stop feature may comprise a radially inwardly extending protrusion. Generally, the radial protrusion may comprise a pin or a flange.

According to another example the preselector stop feature comprises a radial protrusion protruding from a sidewall of the preselector. The preselector may also comprise a sleeve like shape. The preselector stop feature may comprise a radially inwardly extending protrusion to engage with a radially outwardly extending protrusion of the tracking stop feature. Alternatively, the radial protrusion of the preselector stop feature may comprise a radially outwardly extending protrusion to engage with a radially inwardly extending protrusion of the tracking stop feature.

This applies in particular where the tracking stop feature is provided on the housing of the injection device. The radial protrusion of the preselector stop feature may comprise a pin or a flange. Typically, the at least one radial protrusion of the tracking stop feature and the at least one radial protrusion of the preselector stop feature are correspondingly or complementary shaped. They are arranged on a particular portion or section of the dose tracker or housing and the preselector such that upon reaching the maximum dose positional state the radial protrusions of the tracking stop feature and the preselector stop feature get in direct mechanical abutment thereby impeding any further displacement of the dose tracker relative to the housing in a dose incrementing direction.

According to another example one of the preselector stop feature and the tracking stop feature comprises at least a first groove and a second groove configured to slidably receive the radial protrusion of the other one of the preselector stop feature and the tracking stop feature. It is intended that a radial protrusion of one of the preselector stop feature and the tracking stop feature slides along only one of the first groove and the second groove of the other one of the preselector stop feature and the tracking stop feature. When the preselector is in a first of the two preselection positional state the radial protrusion slides along the first groove. When the preselector is in a second of the at least two preselection positional states the radial protrusion slides along the second groove.

The first and the second grooves comprise different shapes. In this way, the first and the second grooves enable and provide different relative displacement paths between the preselector and the dose tracker or the housing. Likewise, the differently shaped grooves provide different displacement path length of the dose tracker between the zero dose positional state of the maximum dose positional state relative to the preselector or relative to the housing, respectively.

In a further example the first groove extends parallel to the second groove. The second groove is longer than the first groove. The first groove and the second groove merge into a connecting groove. The connecting groove extends along a direction that is substantially parallel to that direction along which the preselector is displaceable between the first preselection positional state and the second preselection positional state. While in the zero dose positional state the protrusion of the tracking stop feature may be located in the connecting groove. A displacement of the preselector along the elongation of the connecting groove leads to a sliding motion of the protrusion along the connecting groove.

When reaching one of the at least two preselection positional state the protrusion is still in the connecting groove but is also aligned with one of the first groove and the second groove. When the preselector is in the first preselection positional state the protrusion is aligned with the first groove. When the preselector is arranged in the second preselection positional state the protrusion is aligned with the second groove. As soon a dose setting procedure is started the protrusion slides along the first groove or along the second groove depending on the preselection positional state of the preselector.

The first and the second groove comprise a stop for the protrusion at an end facing away from the connecting groove. Since the first and second grooves are of different length they provide different displacement paths for the radial protrusion sliding along the respective groove. In this way differently sized maximum dose positional states of the dose tracker can be provided. Once the radial protrusion reaches an end of a respective groove facing away from the connecting groove any further displacement of the dose tracker relative to the housing is effectively impeded.

According to another example the preselector is rotationally supported on the housing. Alternatively, the preselector is displaceable relative to the housing along a tangential or circumferential direction. The preselector may comprise a preselector sleeve. The preselector sleeve may be located at a proximal end of the housing. The preselector sleeve may be provided between a proximal portion of the dose tracker and a proximal end of the housing. The preselector may be located directly on or may be supported by the housing at a predefined distance from the proximal end of the housing. Here, the preselector may be located and arranged at a predefined distal offset from the proximal end of the housing.

In another example the preselector may be translationally fixed to the dose tracker but may be freely rotatable relative to the dose tracker. Here, the preselector may be in keyed engagement or may be splined to the housing, e.g. by way of one of the first and second grooves of the preselector stop feature engaged with the tracking stop feature.

Generally and in some examples the preselector is arrestable or fixable to the sidewall of the housing in at least two different discrete positions denoted as preselection positional states. The preselection positional states may be equidistantly arranged on the sidewall. A distance between neighboring preselection positional states is identical and corresponds to the longitudinal advancing motion of the dose tracker as the dose tracker may undergo a complete revolution relative to the housing. In this way it is guaranteed, that the tracking stop feature always exactly engages with the preselector stop feature when reaching the maximum dose positional state.

According to a further example the injection device comprises a trigger and a piston rod. The trigger is arranged at a proximal end of the dose tracker. The trigger may be effectively locked to the dose tracker in longitudinal direction. Hence, any displacement of the dose tracker in longitudinal direction is equally transferred to the trigger. For initiating or for triggering a dispensing procedure the trigger is depressible in a distal direction to induce a distally directed motion of the piston rod. The injection device may further comprise a dose dial that is also translationally fixed to the dose tracker. By means of the dose dial the dose setting may be conducted and/or controlled especially for injection devices that are void of a spring driven mechanical drive.

Typically, the injection device comprises at least one clutch by way of which the dose setting mechanism or drive mechanism can be switched between a dose setting mode and a dose dispensing mode. The clutch may be operable by a depression of the trigger relative to the housing and/or relative to the dose tracker.

In another example the injection device further comprises a cartridge. The cartridge comprises a barrel filled with the medicament. The barrel is sealed by a bung or piston that is axially displaceable relative to the barrel by means of the piston rod. For and during a dispensing operation the piston rod is operably engageable with the bung of the cartridge in order to displace the bung in a distal direction. Typically, a distal end of the cartridge is sealed by a pierceable membrane, such as a septum. For dispensing of the medicament the pierceable seal is penetrated by a double-tipped injection needle. A distally directed displacement of the bung induced by a correspondingly advancing piston rod therefore leads to the expelling of the dose of the medicament.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,
wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present injection device without departing from the spirit and scope what is disclosed herein. Further, it is to be noted, that any reference numerals used in the appended claims are not to be construed as limiting the scope of the invention.

### BRIEF DESCRIPTION OF THE FIGURES

In the following, embodiments of the drive mechanism and the injection device are described in detail by making reference to the drawings, in which:

FIG. 1 is illustrated of an example of an injection device,

FIG. 2 shows an exploded view of the components of the injection device,

FIG. 3 is an exemplary and simplified illustration of the injection device with the dose tracker in the zero dose positional state,

FIG. 4 is indicative of the device according to FIG. 3 with the dose tracker in the maximum dose positional state,

FIG. 5 shows a longitudinal cross-section of some components of the injection device according to FIG. 3,

FIG. 6 shows a longitudinal cross-section of the device according to FIG. 4,

FIG. 6a shows a longitudinal cross-section of a modification of the device according to FIG. 3,

FIG. 6b shows a longitudinal cross-section of the device of FIG. 6a with the clutch and the preselector shifted in distal direction,

FIG. 7 is illustrative of a longitudinal cross-section of another example of an injection device according to FIG. 3,

FIG. 8 shows the injection device of FIG. 7 with the dose tracker in the maximum dose positional state,

FIG. 9 shows a further example of an injection device with the dose tracker in the zero dose positional state,

FIG. 10 shows the device according to FIG. 9 with the dose tracker in the maximum dose positional state,

FIG. 11 is an exemplary illustration of another injection device with a preselector located on a housing of the injection device with the dose tracker in the zero dose positional state,

FIG. 12 shows the device according to FIG. 11 with the dose tracker in the maximum dose positional state,

FIG. 13 is a longitudinal cross-section through a device highly similar to the device according to FIG. 11,

FIG. 14 shows the device of FIG. 13 with the dose tracker in the maximum dose positional state,

FIG. 15 is a further illustration of an injection device, wherein the preselector is translationally locked to the dose tracker,

FIG. 16 shows the injection device according to FIG. 15 with the dose tracker in the maximum dose positional state,

FIG. 17 shows a longitudinal cross-section through the device according to FIG. 15,

FIG. 18 shows a longitudinal cross-section of the device according to FIG. 16,

FIG. 19 is a perspective view of a further example of an injection device comprising a dose tracker and a preselector,

FIG. 20 shows the injection device according to FIG. 19 with the dose tracker in the maximum dose positional state,

FIG. 21 shows the device according to FIGS. 19 and 20 without the outer housing,

FIG. 22 shows the injection device according to FIG. 21 with the preselector,

FIG. 23 is an isolated illustration of a release member,

FIG. 24 shows an interaction between the dose tracker and the release member before reaching the end of a dispensing procedure,

FIG. 25 is an illustration in accordance to FIG. 24 with the dose tracker moved even closer to the zero dose positional state,

FIG. 26 shows the interaction of the release member and the dose tracker when reaching the zero dose positional state and

FIG. 27 shows the mutual interaction of the release member and the dose tracker 40 and the dose tracker in the zero dose positional state,

FIG. 28 is a longitudinal cross-section through the device according to FIG. 19,

FIG. 29 is illustrative of a further longitudinal cross-section through the device of FIG. 19 rotated by 90° with regards to a longitudinal axis of the injection device,

FIG. 30 is exemplary of the interaction between an interlock and a release member in an initial configuration,

FIG. 31 shows the arrangement of FIG. 30 with the interlock released,

FIG. 32 shows another example of an interaction between an interlock and a release member in an initial or interlocked configuration and

FIG. 33 shows the arrangement of FIG. 32 with the interlock released.

### DETAILED DESCRIPTION

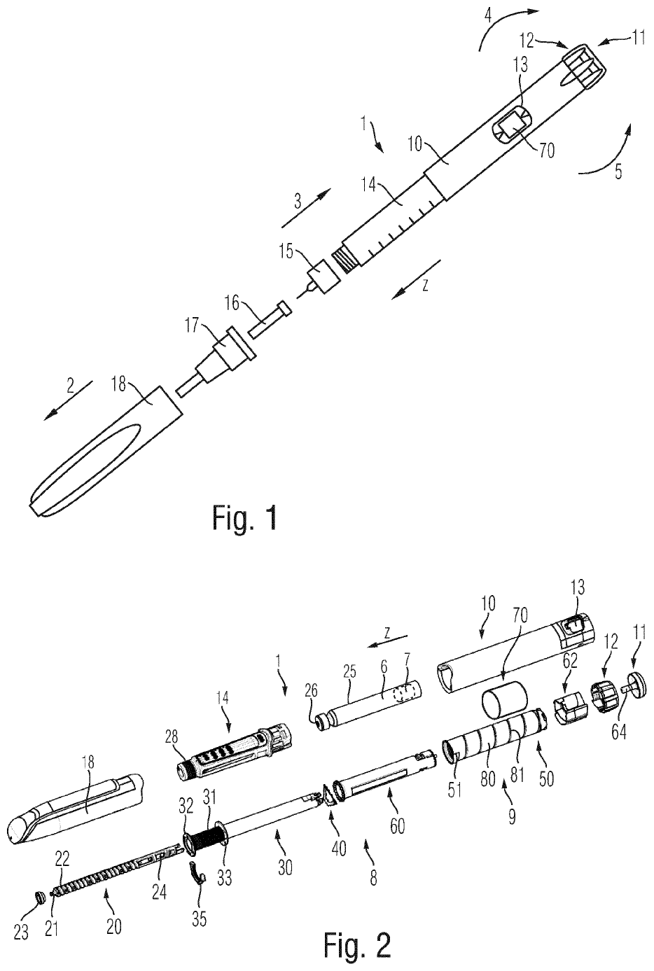

The injection device 1 as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 may comprise and form a main housing part configured to accommodate a drive mechanism 8 as shown in FIG. 2. The injection device 1 may further comprise a distal housing component denoted as cartridge holder 14. The cartridge holder 14 may be permanently or releasably connected to the main housing 10. The cartridge holder 14 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder 14 comprises a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg).

As shown further in FIGS. 1 and 2, the housing 10 comprises a dosage window 13 that may be in the form of an aperture in the housing 10. The dosage window 13 permits a user to view a limited portion of a number sleeve 80 that is configured to move when the dose dial 12, e.g. in form of a dose dial button or dose dial sleeve is turned, to provide a visual indication of a currently set dose. The dose dial is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 will be ejected from the injection device 1. When the needle 15 of the injection device 1 remains for a certain time in the skin portion after the trigger 11 is pushed, a high percentage of the dose is actually injected into the patient's body. Ejection of a dose of the medicament may also cause a mechanical click sound, which is however different from the sounds produced when using the dose dial 12.

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

The expelling or drive mechanism 8 as illustrated in more detail in FIG. 2 comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange. The clutch 60 may comprise a clutch sleeve.

Furthermore, there is provided a dose dial sleeve also denoted as a number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a protrusion 63 or helical rib at an inside sidewall portion of an insert piece 62, and the helical rib is to be seated in the helical groove 81 of the number sleeve 80. The tubular shaped insert piece 62 is inserted into the proximal end of the housing 10. It is rotationally and axially fixed to the housing 10. There may be provided first and second stops on the housing 10 to limit a dose setting procedure during which the number sleeve 80 is rotated in a helical motion relative to the housing 10. As will be explained below in greater detail, at least one of the stops is provided by a preselector stop feature 71 provided on a preselector 70.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement therebetween. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To set or to dial a dose a user rotates the dose dial 12. With the spring 40 also acting as a clicker and the clutch 60 engaged, the drive sleeve 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 provided on the preselector 70 to prevent further movement in a dose incrementing direction 4. A rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated, in the dose decrementing direction 5. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

Compared to the injection device as described in any one of the documents WO2004/078239A1, WO 2004/078240A1 or WO 2004/078241A1 the injection device according to FIGS. 1 and 2 is further provided with the preselector 70. The preselector 70 is displaceable relative to the housing 10 between at least two preselection positional states in order to define a maximum 54 dose positional state of the dose tracker 50. In the example of FIG. 2 the dose tracker 50 may comprise a number sleeve 80 having a helical groove 81 that is in threaded engagement with the housing 10 or with the insert 62 that is fixed to the housing 10.

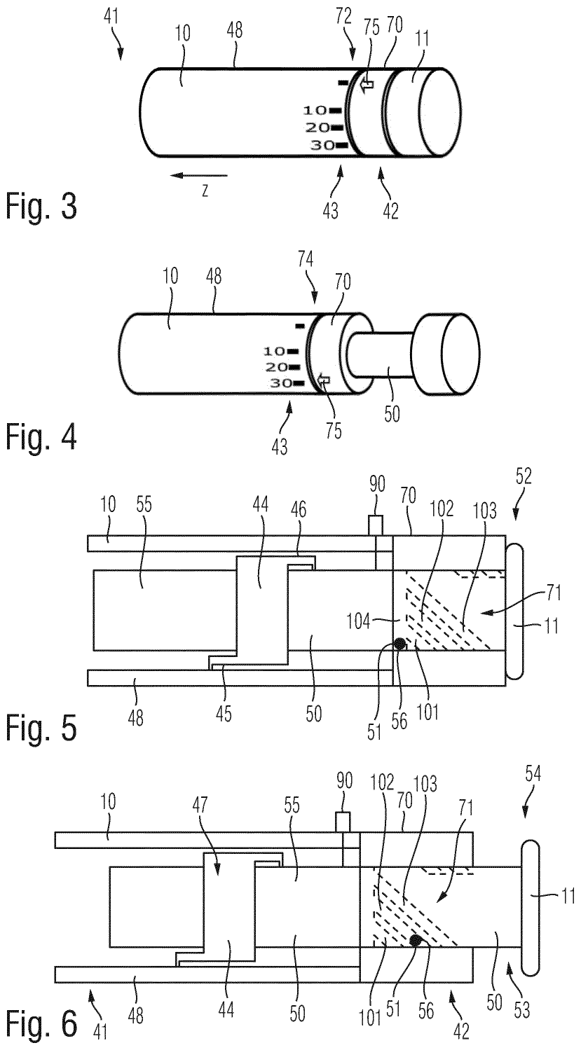

On an outside surface of the number sleeve 80 there may be provided consecutive numbers that show up in the dosage window 13. Selection and indication of visualization of a dose is modified with the various examples of an injection device as described hereinafter with regards to FIGS. 3 to 29. With the various examples as illustrated in FIGS. 3 to 29 the number sleeve 80 provided by the dose tracker 50 is displaceable in unison with a trigger 11 relative to the housing 10 for setting as well as for dispensing of the dose of the medicament.

In the example of FIGS. 3 to 6 there is provided a preselector 70 that is displaceable relative to the housing 10 between at least two preselection positional states 72 and 74. Each preselection positional state 72, 74 defines a maximum dose positional state 54 for the dose tracker 50. In the present example the preselector 70 comprises a preselector sleeve that is rotationally fixed to the tubular shaped housing 10.

As shown in FIGS. 3 to 6 the preselector 70 is provided and rotationally supported at a proximal end 42 of the housing 10. It may be rotationally supported on a side wall 48 of the housing 10. For selecting at least one of two available preselection positional states 72, 74 the preselector 70 is rotatable with regard to a rotation axis extending parallel to the longitudinal axis of the housing 10. The preselector 70 is lockable or fixable relative to the housing 10 in any one of the at least two preselection positional states 72, 74. In this way and when the preselector 70 is in a first preselection positional state the preselector is hindered and impeded against self actuated displacement relative to the housing.

The preselector 70 comprises a preselector stop feature 71. The preselector stop feature 71 as illustrated in FIG. 5 comprises a first groove 101 and a second groove 102. The grooves 101, 102 are provided on an inside facing surface of the sleeve of the preselector 70. The dose tracker 50 comprises a tracking stop feature 51. The tracking stop feature comprises a radial protrusion 56 protruding radially outwardly from an outside surface of the dose tracker 50. Here, the dose tracker 50 comprises a tracking sleeve 55 that is rotationally and translationally supported inside the elongated housing 10.

Typically, the dose tracker 50 is in threaded engagement with the housing 10. As illustrated in FIG. 5 and when in the zero dose positional state the tracking stop feature 51 is located inside a connecting groove 104 interconnecting the first groove 101 and the second groove 102. One end, e.g. a first end of the first groove 101 merges into the connecting groove 104. A first end of the second groove 102 also merges into the connecting groove 104. The connecting groove 104 extends at a predefined angle relative to the elongation of the first groove 101 and the second groove 102. Typically, first and second grooves 101, 100 extend parallel to each other. As illustrated, the second groove 102 comprises a larger extension compared to the first groove 101. There is also provided a third groove 103. Also the third groove 103 extends parallel to the first groove 101 and to the second groove 102. The third groove 103 comprises an elongation that is larger than the elongation of the second groove 102. As shown further, the second groove 102 is located between the first groove 101 and the third groove 103.

The connecting groove 104 comprises an elongation that aligns with and/or coincides with a direction of displacement of the preselector 70 when the preselector is displaced between the at least two preselection positional states 72, 74. For transferring and displacing the preselector 70 from the first preselection positional state 72 as illustrated in FIG. 3 to the second preselection positional state 74 as illustrated in FIG. 4 the preselector 70 is rotatable relative to the housing 10, e.g. in a counterclockwise direction. Accordingly, the connecting groove 104 extends in circumferential or tangential direction with regards to the tubular shaped housing 10 or with regards to the tubular shaped preselector 70.

As further illustrated in FIGS. 3 and 4, the housing 10, in particular the sidewall 48 thereof is provided with a preselection indication 43. The preselection indication 43 comprises numerous numbers or symbols arranged along a displacement path of the preselector 70. The preselector 70 comprises a correspondingly shaped preselection indication 75, e.g. in form of an arrow. In each one of the provided preselection positional states 72, 74 the preselection indication 75 of the preselector 70 aligns with one of the preselection indications 43 of the housing 10.

In an alternative implementation, the preselection indication 43 comprises a pointer or an arrow and wherein the preselection indication 75 comprises numerous numbers or symbols arranged along a displacement path of the preselector 70. The preselection indication 75 aligning with a preselection indication 43 indicates to the user, which one of the preselection positional states 72, 74 is actually valid for the injection device. In the present example there may be provided three or even four preselection positional states. In a first preselection positional state the tracking stop feature 51 is in alignment with the first groove 101. In a second preselection positional state the tracking stop member 51 is in alignment with the second groove 102.

Optionally, there is provided a release member 90 that is connected to one of the housing 10 and the dose tracker 50. It is selectively engageable to the other one of the housing 10 and the dose tracker 50 in order to lock the dose tracker 50 to the housing 10 when in the zero dose positional state 52 as illustrated in FIG. 5. In the present example there is further provided a mechanical energy reservoir in form of a spring 44. The spring 44 comprises a first end 45 connected to the housing 10 and the spring 44 comprises a second end 46 connected to the dose tracker 50. If the release member 90 is actuated in order to liberate or to release the dose tracker 50 the dose tracker 50 starts to rotate relative to the housing 10 under the action of the relaxing spring 44.

As illustrated the spring 44 comprises a cylindrically wound torsion spring 47. The spring 44 encloses at least a portion of an outside surface of the tracking sleeve 55 of the dose tracker 50. In this way and when released the spring 44 is configured to induce a torque to the dose tracker 50.

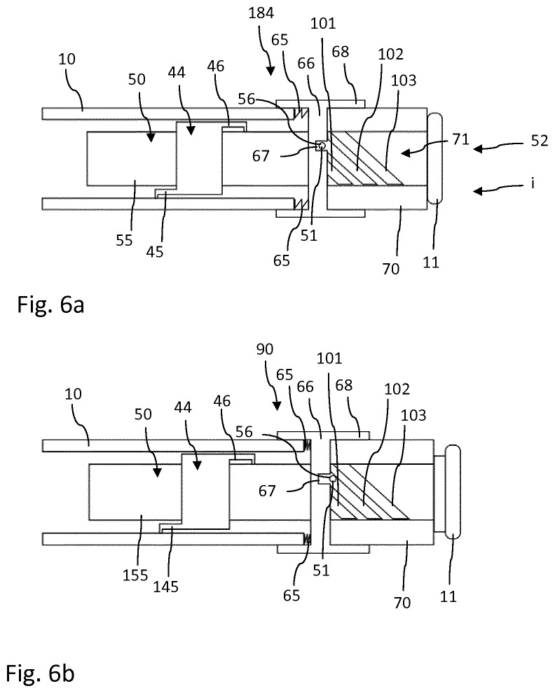

In the given preselection positional state 72, 74 the preselector 70 is rotationally fixed to the housing 10. Here, the engagement of the tracking stop feature 51 with one of the grooves 101, 102, 103 provides a threaded engagement between the dose tracker 50 and the housing 10. Since the preselector 70 is translationally or axially fixed to the housing 10 the dose tracker 50 is subject to a proximally directed displacement such that a proximal end 53 of the dose tracker protrudes from a proximal end of the preselector 70 and/or from a proximal end 42 of the housing 10 when reaching the maximum dose positional state 54 as illustrated in FIG. 6.

The amount of displacement or the length of a displacement path of the dose tracker 50 relative to the housing 10 is indicative and is directly correlated to the size of a dose actually set. The grooves 101, 102, 103 each comprise a second end facing away from the connecting groove 104. The second end of the grooves 101, 102, 103 provides an end stop for the tracking stop feature 51. Once the tracking stop feature 51, presently in form of a radially outwardly extending protrusion 56, reaches the second end of the second groove 102 as illustrated in FIG. 6 or 8 further displacement of the dose tracker 50, 150 in a dose incrementing direction relative to the preselector 70 and/or relative to the housing 10 is effectively impeded and blocked.

Once the maximum dose positional state 54 has reached the injection device 1 is prepared and ready for a dose dispensing procedure. For this, a user has to depress the trigger 11 in distal direction as described above with regard to FIGS. 1 and 2. During a dispensing procedure the dose tracker 50 returns into the zero dose positional state 52. It rotates in a dose decrementing direction 5 relative to the housing 1 in accordance and along the helical path provided by the respective grooves 101, 102 or 103. When reaching the zero dose positional state 52 as illustrated in FIGS. 3 and 5 the release member 90 reengages and positionally fixes the dose tracker 50 to the housing 10. Thereafter, the preselector 70 may be transferred to another preselection positional state in order to vary the size of the dose if required. Otherwise, the preselector 70 remains in the present preselection positional state. A repeated release of the release member 90 will lead to another automated displacement of the dose tracker 50 from the zero dose positional state 52 to the maximum dose positional state 54. Accordingly, another dispensing procedure may take place.

The implementation of the spring 44 and the automated displacement of the preselector 50 from the zero dose positional state 52 to the maximum dose positional state 54 is only optional. Alternatively and when the injection device 1 is void of such a driving spring 44 the displacement of the dose tracker 50 from the zero dose positional state 52 to the maximum dose positional state 54 is governed and conducted by manually rotating the dose dial 12 in the dose incrementing direction 4, e.g. clockwise to the housing 10.

In the example of FIGS. 3 to 6 the dose tracker 50 may also comprise not only one but even two or more radially outwardly extending protrusion 56 that are arranged circumferentially offset, e.g. at 180°. Accordingly the preselector 70 may comprise a respective amount of grooves, e.g. 6 grooves altogether. Here, two diametrically oppositely located protrusions 56 of the dose tracker 50 may be always and simultaneously in engagement with two oppositely located grooves of the preselector 70.

In the example of FIGS. 3 to 6 the dose tracker 50 may be in threaded engagement with the housing 10 only via the tracking stop feature 51 sliding along the preselector stop feature 71 of the preselector 70. It is generally conceivable, that the insert 62 as described in connection with FIGS. 1 and 2 is replaced by the preselector 70. In this way, only minor modifications have to be implemented in the injection device 1 as described in any of the documents WO2004/078239A1, WO 2004/078240A1 or WO 2004/078241A1 in order to implement a preselection of only a limited number of different dose sizes.

In FIGS. 6a and 6b a modification of the device as illustrated in FIGS. 3-6 is illustrated. Here, the injection device is equipped with a supplemental clutch 66 having a recess 67 to engage with the protrusion 56 and hence with the tracking stop feature 51 of the dose tracker 50. The supplemental clutch 66 may comprise a clutch sleeve. The supplemental clutch 66 may comprise a tubular shaped body with a tubular-shaped sidewall 68. The clutch 66 is attached to the housing 10. It may be located on an outside surface of the housing 10. It may be displaceable in longitudinal direction relative to the housing 10. The clutch 66 is rotationally fixed to the housing 10. The clutch 66 may be in a splined engagement with the housing 10. Hence, the clutch 66 is hindered to rotate relative to the housing 10. The clutch 66 may be in a longitudinally sliding and rotation inhibiting engagement with the housing 10.

In the zero dose positional state 52 of the dose tracker 50 as illustrated in FIG. 6a the tracking stop feature 51 of the dose tracker 50 and hence the protrusion 56 is located inside the recess 67 of the clutch 66. The recess 67 comprises a tangential or circumferential width that substantially matches with the respective size or width of the protrusion 56. The width or size of the recess 67 may be slightly larger than the size of the protrusion so as to enable a smooth insertion of the protrusion 56 into the recess 67. The recess 67 is open towards the proximal direction 3.

The clutch 66 is axially displaceable in distal direction 2 against the action of a spring 65. One end of the spring 65 is engaged with the clutch 66 and the opposite end of the spring 65 is engaged with the housing 10. The spring 65 may comprise a compression spring. It may be configured to urge or to drive the clutch 66 in and towards the proximal direction 3. As long as the protrusion 56 is located inside the recess 67 the mutual engagement of the protrusion 56 and the recess 67 hinders the dose tracker 50 from rotating under the action of the spring 44.

The position of the recess 67 matches and overlaps with the position of the protrusion 56 as the dose tracker 50 is in the zero dose positional state 52. By depressing the clutch 66 in distal direction the recess 67 is moved in distal direction accordingly. As a consequence, the protrusion 56 is no longer retained inside the recess 67 and the dose tracker 50 becomes free to rotate under the action of the spring 44.

The preselector 70 is axially engaged with the clutch 66. It is fixed to the clutch 66 in axial or longitudinal direction. Any movement of the clutch 66 in longitudinal or axial direction equally transfers to a respective movement of the preselector 70. The preselector 70 is rotatable relative to the clutch 66. In any of its rotational states, the preselector 70 is rotationally fixable to the clutch and hence to the housing 10. The preselector 70 may be in a kind of a snap-fit engagement or ratchet engagement with the housing 10 or with the clutch 66. This allows and supports a dedicated rotation of the preselector 70 with the longitudinal axis of the injection device as an axis of rotation, so as to bring one of the groves 101, 102, 103 in axial or longitudinal alignment with the tracking stop feature 51 as the tracking stop feature is in the zero dose positional state. The rotation of the preselector 70 relative to the housing 10 and/or relative to the clutch 66 may be accompanied by an audible click sound or haptic feedback.

When in the zero dose positional state 52 the preselector 70 is rotatable relative to the housing 10 as well as relative to the clutch 66 in order to preselect a dose of a particular size. For instance and as illustrated in FIG. 6a the second groove 102, in particular the distal end of that groove 102, is brought in longitudinal alignment with the recess 67 and hence with the protrusion 56 or tracking stop feature 51 located therein.

Since the preselector 70 is axially connected to the clutch 66, a distally directed displacement of the clutch 66 equally transfers to a respective distally directed displacement of the preselector 70; and vice versa. As a consequence, the tracking stop feature 51 and hence the protrusion 56 slides out of the recess 67 and enters the preselector stop feature 71, i.e. the groove 102. Through this axial displacement of the preselector 70 relative to the housing 10 the protrusion 56 enters the groove 102. The protrusion 56 is then allowed to slide along the helical path provided by the groove 102. In this way the entire dose tracker 50 becomes subject to a proximally directed screwing motion relative to the housing 10 as it is free to rotate under the action of the spring 44 as described above in connection with FIGS. 3-6.

At the end of a dose delivery procedure during which the dose tracker 50 is moved in distal direction 2 and during which the dose tracker 50 returns into the zero dose positional state 52 the tracking stop feature 51 and hence the protrusion 56 re-enters the recess 67. As the dose dispensing or injection procedure terminates the mutual engagement of the tracking stop feature 51 or protrusion 56 with the recess 67 hinders the dose tracker 50 from rotating.

In the example of FIGS. 6a and 6b the release member 90 may be replaced by the clutch 66. Here, the clutch 66 may provide both, an interlock 184 as well as a release member 90. In the proximal position as indicated in FIG. 6a the clutch 66 provides an interlock 184 configured to prevent a rotation of the dose tracker 50 relative to the housing 10. In the distal position as indicated in FIG. 6b the clutch 66 provides a release member 90 disengaging the interlock 184, thus allowing and supporting a rotation of the dose tracker 50 relative to the housing 10.

The clutch 66 as illustrated in FIGS. 6a and 6b may be integrally formed with the clutch 60 as illustrated in FIG. 2. The clutch 66 may be a portion of the clutch 60. In further examples the clutch 66 and the clutch 60 may be separate parts.

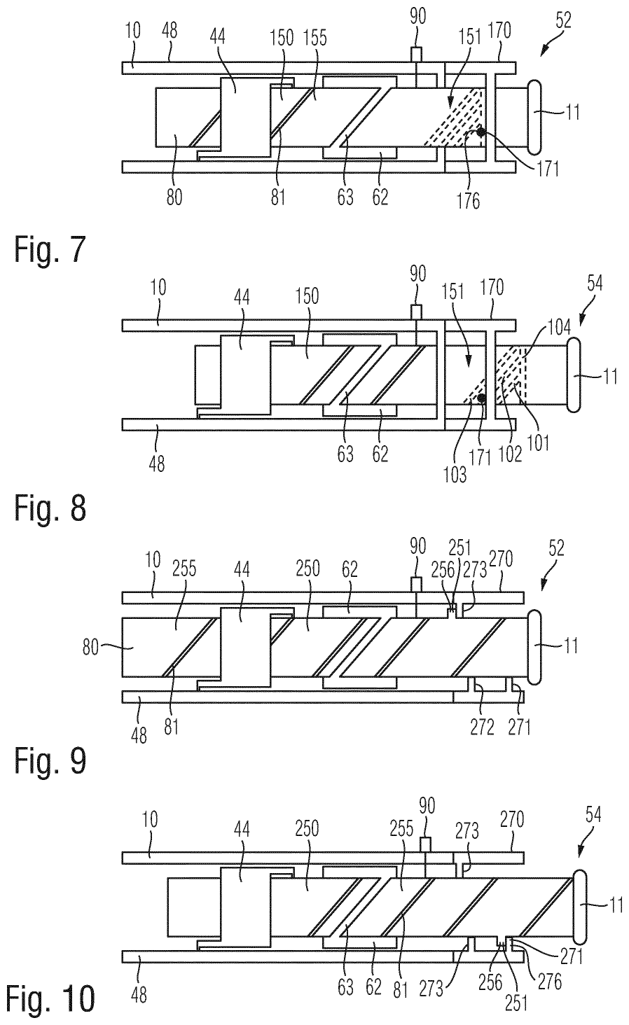

In FIGS. 7 and 8 another example of an injection device 1 is illustrated. Identical or components compared to the example of FIGS. 3 to 6 are denoted with identical reference numbers. Similar components compared to the example of FIGS. 3 to 6 are denoted with respective reference numbers that are increased by 100.

In comparison to the example of FIGS. 5 and 6 the example of FIGS. 7 and 8 comprises an insert 62 that is fixed to the housing 10. The insert 62 is typically fixed to a sidewall 48 of the housing 10. The insert 62 is a threaded insert. The insert 62 comprises a radially inwardly extending protrusion 63. The protrusion 63 may comprise a helical shape. It may be in threaded engagement with an outer thread or with a helical groove 81 on the outside surface of the dose tracker 150. Also here the preselector 170 is of sleeve-like shape. It is rotationally supported at or near a proximal end of the housing 10. The preselector 170 comprises a preselector stop feature 171. The dose tracker 150 comprises a correspondingly shaped tracking stop feature 151. Compared to the example of FIGS. 5 and 6 it is the tracking stop feature 151 that comprises a first groove 101, a second groove 102, a third groove 103 and a connecting groove 104. The preselector stop feature 171 comprises a radial protrusion 176.

The radial protrusion 176 may protrude radially inwardly from the sleeve-shaped preselector 170. The radial protrusion 176 is in sliding engagement with one of the grooves 101, 102, 103, 104. As the dose tracker 150 is subject to a rotation due to the threaded engagement with the insert 62 the radial protrusion 176 slides along the elongation of one of the grooves 101, 102, 103. Also here, the dose tracker 150 may comprise two or even more preselector stop features 171, e.g. in form of two or even more radial protrusions 176 that are simultaneously engaged with a corresponding number of grooves.

In the zero dose positional state 52 as illustrated in FIG. 7 the preselector stop feature 171 is slidably engaged with the connecting groove 104. A rotation of the preselector 170 relative to the housing 10 provides an alignment of the preselector stop feature 171 with one of the grooves 101, 102, 103. After a release of the dose tracker 50 by actuating the release member 90 the dose tracker 50 starts to rotate according to the threaded engagement with the insert 62 relative to the housing 10 and hence relative to the preselector 170, which is rotationally fixed to the housing 10 in the respective preselection positional state.

As illustrated in FIG. 8 the preselector stop feature 171, in particular a radially inwardly extending protrusion 176 as provided on an inside facing sidewall of the sleeve-shaped preselector 170 slides along the groove 102 until it reaches a second end of the groove providing a stop for the preselector stop feature 71. In this maximum dose positional state 54 any further proximally directed displacement of the dose tracker 50 is blocked through the mutual engagement of the protrusion 176 with the second end of the groove 102. The mode of operation of the device according to FIGS. 7 and 8 is comparable if not identical to the mode of operation as described in connection with the device according to FIGS. 3 to 6.

The further example of FIGS. 9 and 10 is somehow similar to the example as described in connection with FIGS. 7 and 8. Also here, the dose tracker 250 comprises a tracking stop feature 251. The dose tracker 250 comprises a tracking sleeve 255 that is in threaded engagement with the insert 62. Also here, the preselector 270 is of sleeve-like shape. It is also longitudinally or axially fixed to the housing 10, in particular to a sidewall 48 thereof. The preselector 270 is displaceable supported on the housing 10 or relative to the housing between at least two preselection positional states 72, 74.

The preselector 270 comprises a preselector stop feature 271, which is implemented as a radial protrusion 276 protruding radially inwardly from a sidewall of the preselector 270. The correspondingly shaped tracking stop feature 251 of the dose tracker 250 is provided on an outside surface portion of the tracking sleeve 255. The tracking stop feature 251 comprises a radially outwardly extending protrusion 256. For setting of a dose and for transferring the dose tracker 250 from the zero dose positional state 52 to the maximum dose positional state 54 the dose tracker 250 rotates in accordance to the threaded engagement with the housing 10.

The preselection positional state of the preselector 270, hence the orientation of the preselector 270 with regard to a rotation axis thereof defines the positional state, hence the longitudinal position and/or an orientation of the dose tracker 250 relative to the housing 10 at least when the tracking stop feature 251 abuts with the preselector stop feature 271. As illustrated in FIGS. 9 and 10 the preselector 270 may comprise numerous preselector stop features 271, 272 and 273. The various preselector stop features 271, 272, 273 all comprise a radially inwardly extending protrusion 276. The various preselector stop features 271, 272, 273 are located at predefined positions at an inside facing sidewall portion of the preselector 270.

The preselector stop features 271, 272, 273 are located at predefined and different axial and/or longitudinal positions along the elongation or along the inner circumference of the preselector 270. The preselector stop features 271, 272, 273 may each comprise a flange protruding radially inwardly from the sidewall of the preselector 270. The tangential or circumferential extension of the flange may be larger than the tangential or circumferential extent of the correspondingly shaped tracking stop feature 251. The tangential or circumferential extension of the preselector stop features 271, 272, 273 is shorter than 180°, shorter than 90° or shorter than 45° with respect to the inner circumference of the preselector 270.

In this way and depending on the rotational state of the preselector 270 the tracking stop feature 251 may pass by at least one of the preselector stop features 273 and 272 on its way towards the maximum dose positional state 54. When reaching the maximum dose positional state 54 the tracking stop feature 251 axially and/or tangentially engages with the correspondingly shaped and preselector stop feature 271.

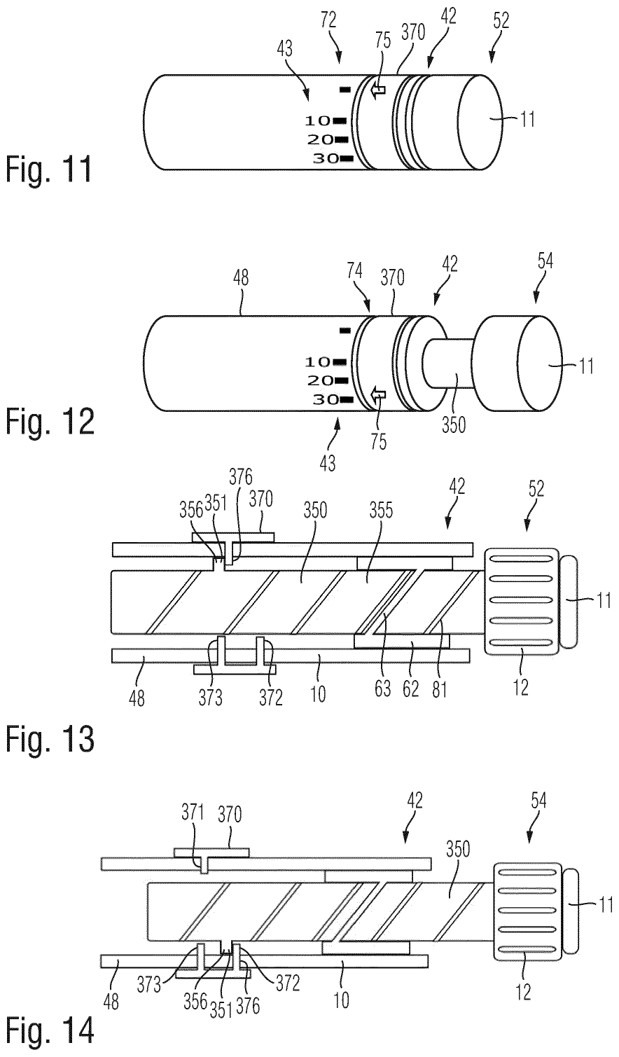

In a further example as illustrated in FIGS. 11 to 14 the preselector 370 is located and arranged at a predefined distance from the proximal end 42 of the housing 10. As illustrated in FIG. 13 the preselector 370 is located distally offset from a proximal end 42 of the housing 10. Apart from that the mechanical interaction between the preselector 370 and the dose tracker 350 is substantially identical as described in connection with FIGS. 9 and 10. Also here, the dose tracker 350 comprises a tracking sleeve 355 that is in threaded engagement with the insert 62 and/or with the housing 10. At or near a proximal end 53 of the dose tracker 350 there is located a dose dial 12 as well as a trigger 11 as described before in connection with FIG. 1 or FIG. 2. The cross-section according to FIGS. 13 and 14 does not exactly match with the perspective illustration of FIGS. 11 and 12. In FIGS. 13 and 14 the preselector 370 is positioned distally offset from the illustration of FIGS. 11 and 12. However, the working principle of the device of FIGS. 11 and 12 is schematically apparent from the cross-sections of FIGS. 13 and 14.

The example of an injection device of FIGS. 11 to 14 is void of a spring 44 configured for automatically displacing the dose tracker 350 from the zero dose positional state 52 towards the maximum dose positional state 54. For displacing the dose tracker 350 from the zero dose positional state 52 towards the maximum dose positional state 54 a user has to grip the dose dial 12 and to rotate the dose dial 12 along a dose incrementing direction as described above. The device according to FIGS. 11 to 14 may be also equipped with a spring 44 as described above.

Also here and as described in connection with FIGS. 9 and 10 the dose tracker 350 comprises a tracking stop feature 351 implemented as a protrusion 356 protruding radially outwardly from an outside surface portion of the tracking sleeve 355. The preselector 370 comprises a sleeve like shape. It is located on an outside surface of the sidewall 48 of the housing 10.

Alternatively, the preselector 370 could be located inside the housing 10. It could be rotationally displaceable in an intermediate space formed between an inside facing surface of the sidewall 48 and an outside facing surface of the dose tracker 350. The preselector 370 comprises at least one preselector stop feature 371. In the illustrated example the preselector 370 comprises numerous preselector stop features 371, 372, 373. The preselector stop features 371, 372, 373 each comprise a radially inwardly extending protrusion 376 in form of a pin or flange. The protrusions 376 may extend through correspondingly-shaped through openings in the sidewall 48 of the housing 10. In another but not illustrated example the protrusion 376 are located entirely inside the housing 10. The preselector 370 may be accessible from outside the housing 10 through a recess or a through opening provided in the sidewall 48.

As already described above the preselector 370 is fixable to the housing 10 or to the sidewall 48 in any of the available preselection positional states. The protrusions 376 comprise a radially inwardly extending pin or a flange having a predefined extension in circumferential or tangential direction, e.g. as described in connection with FIGS. 9 and 10, so as to axially and/or tangentially engage with the correspondingly shaped protrusion 356 of the tracking stop feature 351 when the maximum dose positional state 54 of the dose tracker 350 has been reached.

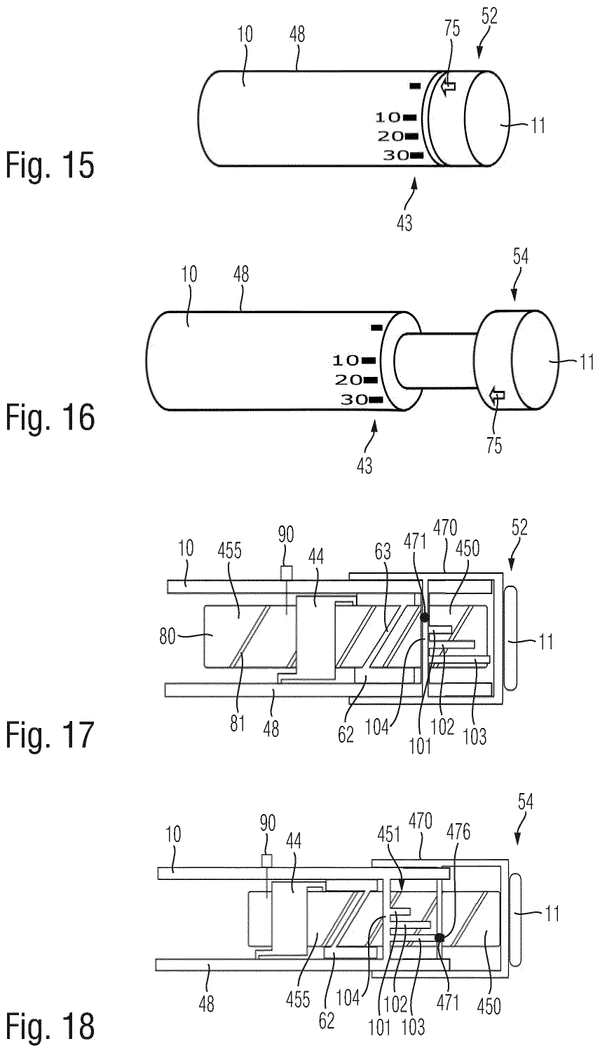

With the further example according to FIGS. 15-18 the preselector 470 is permanently translationally fixed to the dose tracker 450. The preselector 470 is rotatable relative to the dose tracker 450. As already described above in connection with FIGS. 7-14 the dose tracker 450 is threadedly engaged with the housing 10, e.g. via the threaded insert 62. Also here, a spring 44 in form of a torsion spring 47 is provided in order to provide an automated displacement of the dose tracker 450 from the zero dose positional state 52 towards the maximum dose positional state 54.

In the zero dose positional state the dose tracker 450 is positionally locked to the housing 10 by means of the release member 90. In the illustrated example the preselector 470 comprises a sleeve having an inside facing surface that faces towards the outside facing surface of the sidewall 48 of the housing 10. Hence, the preselector 470 comprises a cup-shaped receptacle to receive a proximal end 42 of the housing 10. Other configurations are also conceivable, wherein at least a distal end of the preselector 470 is insertable into the sleeve-shaped housing 10.

In the example as shown in FIGS. 17 and 18 the preselector stop feature 471 provided on the preselector 470 comprises a radially inwardly extending protrusion 476 to engage with the tracking stop feature 451. In contrast to the examples as described above the tracking stop feature 451 is provided on the sidewall 48 of the housing 10. The tracking stop feature 151 comprises a first groove 101, a second groove 102 and a third groove 103. All three grooves 101, 102, 103 merge into a connecting groove 104 with a first end. The grooves 101, 102, 104 comprise different elongations. The grooves 101, 102, 103 extend parallel to each other. The second ends of the grooves 101, 102, 103 are located at a longitudinal offset relative to each other.

In this way the elongation of the groove 101, 102, 103 define the maximum dose positional states 54 of the dose tracker 450. The tracking stop feature 451 is provided on or in an outside facing surface portion of the sidewall of the housing 10. The preselector stop feature 471 protruding radially inwardly from an inside facing section of the sidewall of the preselector 470 is in permanent engagement with at least one of the grooves 101, 102, 103, 104. In the zero dose positional state 52 as illustrated in FIG. 17 the preselector stop feature 471, hence the radial protrusion 476, is located inside the connecting groove 104. By rotating the preselector 470 relative to the housing 10 the preselector stop feature 471 can be aligned with one of the grooves 101, 102, 103. Thereafter and upon releasing of the dose tracker for 450 by actuation of the release member 90 the spring 44 induces a rotation of the dose tracker 450, which according to the threaded engagement with the housing 10 is subject to a helical motion relative to the housing 10.

The grooves 101, 102, 103 extend parallel to the elongation of the housing 10. They extend e.g. perpendicular to the elongation of the connecting groove 104. Since the preselector 470 is freely rotatable relative to the dose tracker 450 but remains axially and longitudinally locked and constrained to the dose tracker 450 the preselector stop feature 471 starts to slide along the selected grooves 103 in the example of FIG. 18 as soon as the dose tracker is subject to a longitudinal movement relative to the housing 10.

The engagement of the preselector stop feature 471 with the groove 103 also prevents a rotation of the preselector 470 relative to the housing 10 during a dose setting motion of the dose tracker 450. When reaching the maximum dose positional state 54, the preselector stop feature 470 gets in abutment with the second end of the groove 103 by way of which a further proximally directed displacement of the preselector 470 is impeded. Due to the permanent longitudinal interlock or engagement between the preselector 470 and the dose tracker 450 any further rotation of the dose tracker 450 is impeded and prevented.

Since the dose tracker 450 is threadedly engaged with the housing 10 any further rotation thereof would require a further displacement in longitudinal direction relative to the housing 10. This is effectively blocked an impeded when the dose tracker 450 is in the maximum dose positional state 54. In the maximum dose positional state 54 as illustrated in FIG. 18 the trigger 11 can be depressed in order to induce a dose dispensing procedure as described above.

Generally, the preselector may be fixed in the preselection positional states at discrete positions relative to the housing or relative to the dose tracker. The supported preselection states may correspond to consecutive and complete revolutions of the dose tracker. Alternatively or additionally it is also conceivable that the dose tracker comprises two or even three tracking stop features to engage with the preselector stop feature. Alternatively, also the preselector may comprise two or more preselector stop features to engage with the tracking stop feature. In this way the maximum dose positional state could be assigned with every half or every third revolution of the dose tracker relative to the housing. Furthermore it is conceivable, that two or more tracking stop features simultaneously engage with correspondingly shaped two or more preselector stop features. In this way the mechanical interaction and robustness of the abutment between the dose tracker and the preselector can be enhanced and increased.

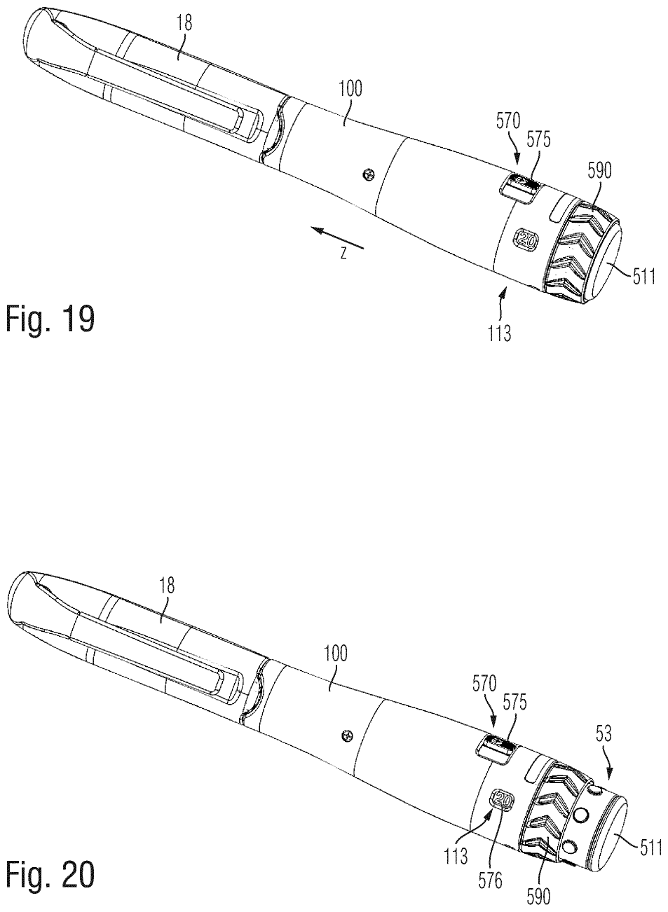

In the further example of an injection device according to FIG. 19 to FIG. 29 the injection device 1 as illustrated in FIG. 1 and FIG. 2 serves as a basis. The injection device as shown in FIG. 19 comprises some additional features as will be explained below in order to provide an enhanced functionality of the injection device 1 as described above.

As illustrated, there is provided an outer housing 100 encapsulating or accommodating the entirety of the housing 10 of the injection device 1. On the outside of the housing 10 there is provided the dose tracker 550. The dose tracker 550 as illustrated in FIG. 22 comprises two components, namely a distal part 552 and a proximal part 553. The distal part 552 and the proximal part 553 may be provided as a single-pieced or as an integrally shaped dose tracker 550. Only for reasons of assembly of the injection device 1 the dose tracker 550 is separated into two separate components.

The distal part 552 and the proximal part 553 are permanently and rigidly connected to each other. They are locked with regards to the longitudinal direction (z) as well as with regard to a rotation relative to the housing 10. A longitudinal displacement or rotational displacement of one of the distal part 552 and the proximal part 553 equally transfers to the other one of the distal part 552 at the proximal part 553.

In the present example the distal part 552 comprises at least one or more elongated ribs 557 extending in longitudinal direction. The ribs 557 provide a keyed and longitudinally sliding engagement with the outer housing 100. The outer housing 100 may comprise a correspondingly shaped longitudinal groove 107 in which the rib or ribs 557 are slidably guided. The dose tracker 550 is rotationally locked to the outer housing 100 but is translationally displaceable relative to the housing 100 in longitudinal or axial direction (z). The dose tracker 550 also comprises a tracking sleeve 555 and a tracking stop feature 551.

As further illustrated in FIG. 22 there is provided a preselector 570 with a preselector stop feature 571. The preselector 570 comprises a sleeve rotatably supported on an outside facing surface of the dose tracker 550. Typically, the distal part 552 and the proximal part 553 of the dose tracking sleeve are of tubular shape. As illustrated in FIGS. 22, 28 and 29 a proximal portion of the distal part 552 is received in a receptacle at a distal portion of the proximal part 553. In the overlapping region the distal part 552 of the proximal part 553 are mutually engaged and permanently interlocked.

The preselector 570 comprises an annular ring or a sleeve with a preselector stop feature 571. As illustrated in FIG. 22 the preselector stop feature 571 comprises numerous axial recesses in a proximal side of the preselector 570. The recesses may form slots of different axial length or of different elongation. The preselector stop feature 571 comprises a first recess 501 and a second recess 502. The recesses 501, 502 comprise different elongations in longitudinal direction as illustrated in FIG. 22. Both recesses 501, 502 are open towards the distal end and hence towards the tracking stop feature 551. The recesses 501, 502 are located tangentially or circumferentially adjacent and next to each other.

Depending on the rotational position of the preselector 570 either the first recess 501 or the second recess 502 longitudinally aligns with the tracking stop feature 551. Since the dose tracker 550 and hence the tracking stop feature 551 thereof can only slide in longitudinal or axial direction relative to the housing and since the preselector 570 is axially or longitudinally fixed to the outer housing 100 the distance between the tracking stop feature 551 and a proximal end of the recesses 501, 502 defines a maximum displacement path for the dose tracker 550 for setting of a dose. Depending on the rotational state, hence depending on the preselection positional state of the preselector 570 the maximum displacement path for the dose tracker 550 can be modified on demand.

The recesses or slots are configured to receive and to engage the tracking stop feature 551 protruding from an outside surface of the tracking sleeve 555. In the present example the tracking stop feature 551 comprises a radially outwardly extending protrusion 556 integrally formed with the distal part 552 and protruding radially outwardly through a correspondingly shaped recess at a sidewall of the proximal part 553. It may likewise be integrally formed with the proximal part 553.

The radial extension of the protrusion 556 matches with the radial extension or radial position of the preselector stop feature 571. The preselector 570 is rotatable between at least two preselection positional states as described above. In any of the preselection positional states the preselector 570 is rotationally locked to the outer housing 100. The preselector 570 is also permanently longitudinally locked to the outer housing 110. For instance, a proximal end 572 or edge of the preselector 570 may be in axial abutment with the outer housing 100 or with another component of the injection device, e.g. with the release member 590 that is axially fixed to the housing 100. In this way the preselector 570 is locked to the outer housing 100 with regard to the longitudinal or axial direction.

The preselector 570 may be further provided with a locking feature 575 extending through a recess or a through opening of the preselector 570. The locking feature 575 may comprise a spring biased actuator that is depressible in radial direction for temporarily releasing the preselector from the outer housing 100. The locking feature 575 may comprise a screw or the like fastening element that requires a correspondingly shaped tool for temporarily releasing the locking feature 575 and hence the preselector 570 from the outer housing 100 in order to enable a sliding motion or rotation of the preselector 570 relative to the outer housing 100. Depending on the selected preselection positional state of the preselector 570 a maximum dose positional state for the dose tracker 550 can be defined.

If the preselector 570 is in a first preselection positional state 74, in which the first recess 501 is longitudinally aligned with the tracking stop feature 551 the maximum distance the dose tracker 550 is longitudinally displaceable relative to the outer housing 100 is shorter compared to a configuration in which the preselector is in the second preselection positional state, in which the second recess 502 is longitudinally aligned with the tracking stop feature 551.

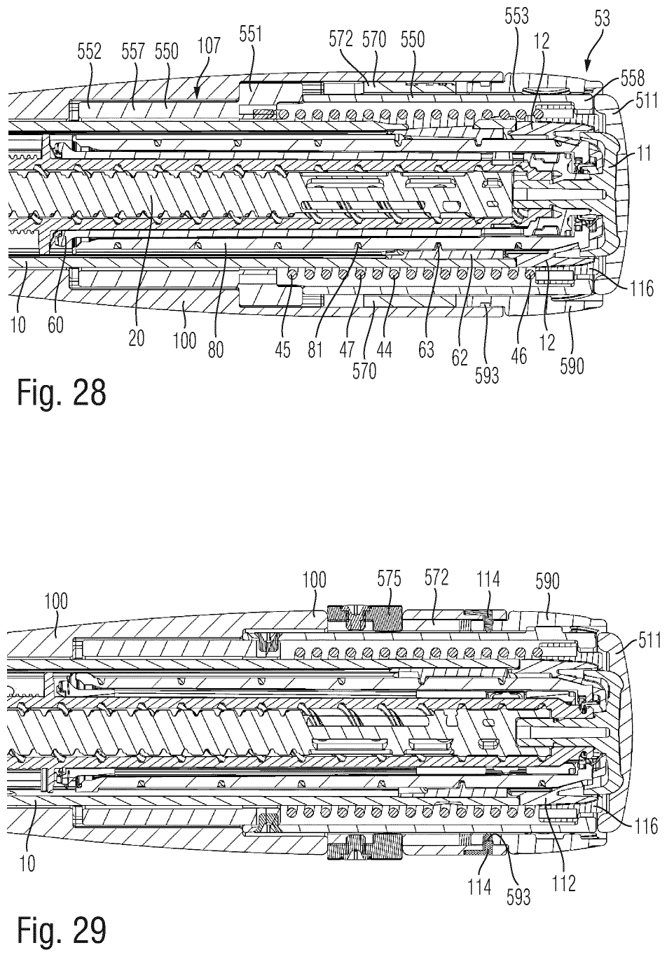

As further illustrated in FIG. 22 there are provided numerous preselection indications 576 on an outside surface portion of the preselector 570. One preselection indication 576 always aligns with a preselection window 130 provided in the outer housing 100. As illustrated in FIGS. 19 and 28 number 20 shows up in the preselection window 113 indicating to the user that a preselection of 20 units of the medicament has been pre-selected. Dialing or displacing the preselector e.g. with the second recess 502 in alignment with the tracking stop feature 551 may reveal a larger number, e.g. number 30 in the preselection window 113.

The interaction between the release member 590 and the dose tracker 550 is illustrated in connection with FIG. 23 to FIG. 27. The release member 590 comprises an annular ring 591 comprising numerous catches 592 at an inside facing portion thereof as illustrated in FIG. 23. The release member 590 comprises an annular groove 593 near a proximal end of the annular ring 591. The groove 593 is positively engaged with a radially inwardly extending fastener 114 at the outer housing 100 as illustrated in FIG. 29. In this way the release member 590 is freely rotatable relative to the outer housing 100 but is permanently locked to the outer housing 100 in longitudinal direction.

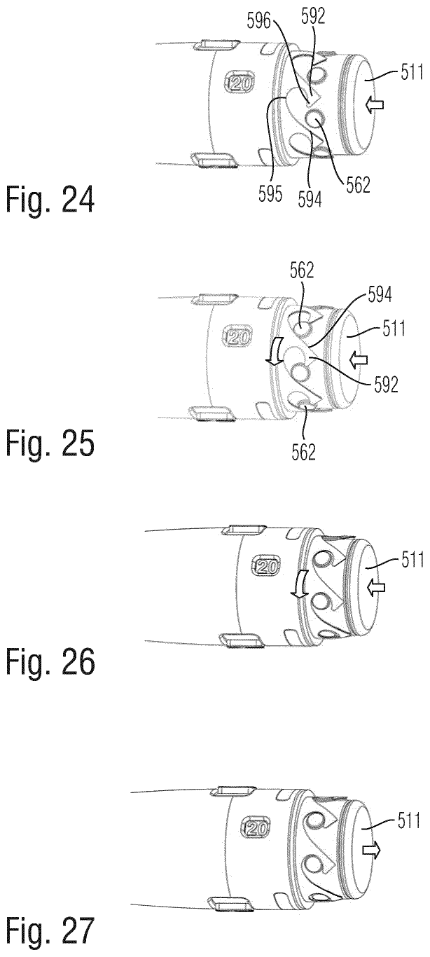

In the sequence of FIGS. 24 to 27 only the catches 592 and the proximal portion of the annular ring 591 are illustrated. An outer section of the annular ring 591 is cut away or faded away for illustration purpose in order to reveal the mutual engagement of the various catches 592 with radially outwardly extending protrusions 562 provided on an outside surface portion of the dose tracker 550. As illustrated, the protrusions 562 are of a pin-shaped structure. They extend radially outwardly near a proximal end of the proximal part 553. The catches 592 and the protrusions 562 are regularly and equidistantly arranged along the outer circumference of the dose tracker 550 and along the inner circumference of the annular ring 591, respectively.

The catches 592 extend at a predefined angle relative to the longitudinal direction. Each catch 592 comprises a rather straight shaped beveled section 594 extending in distal direction into a curved section 595. The curved section 595 extends from the beveled section 594 into the undercut section 596. The curved section 595 may even overlap with the undercut section 596. A free end of the undercut section 596 is located at a predefined tangential or circumferential distance from the beveled section 594. As the protrusion 562 is displaced in distal direction relative to the release member 590 it gets in contact with the beveled section 594 and slides along the beveled section 594 until it reaches the curved section 595 as illustrated by a comparison of FIG. 25 and FIG. 26.

The curved section 595 is shaped and describes at least half of a circle or three-quarter of a circle. It describes a circumference of a circle of about 270°. A bottom of the curved section 595 forms the distal end of the catch 592. Due to the curved section 595 the button thereof is in longitudinal overlapping configuration with the undercut section 596. As the protrusion 562 is displaced in distal direction and returned towards the zero dose positional state 50 the release member 590 is subject to a rotation in accordance to the extension and slope of the beveled section 594 and the curved section 595, respectively. As the protrusion 562 reaches the bottom of the curved section 595 it has tangentially entered a free space between the undercut section 596 and the curved section 595.

Releasing of the trigger 511 in the configuration as shown in FIG. 26 may enable a small spring driven proximally directed displacement of the dose tracker 550. But then the protrusion 562 gets in abutment with the undercut section 596, thereby impeding any further displacement of the dose tracker 550 relative to the release member 590 and hence relative to the outer housing 100 in proximal direction.

For release of the dose tracker 550 the release member 590 has to be rotated in a clockwise direction. In this way, the undercut section 596 induces a slight but distinct initial distal displacement of the dose tracker 550 before the protrusion 562 enters a free space between the undercut section 596 and the beveled section 594 of the catch 592. Due to the regular arrangement of a plurality of catches 592 and protrusions 562 the protrusions 562 and catches 592 mutually engage and disengage simultaneously. Once the protrusions 562 have disengaged from the catches 592 the dose tracker 550 is free to slide in proximal direction relative to the outer housing 100.

The annular ring 591 and hence the release member 590 may be also spring biased, e.g. by a further torsion spring not further illustrated here. In this way, the release member 590 can be kept in an interlocked configuration as shown in FIG. 27. A releasing motion of the release member 590 may have to be conducted against the action of such a return spring.

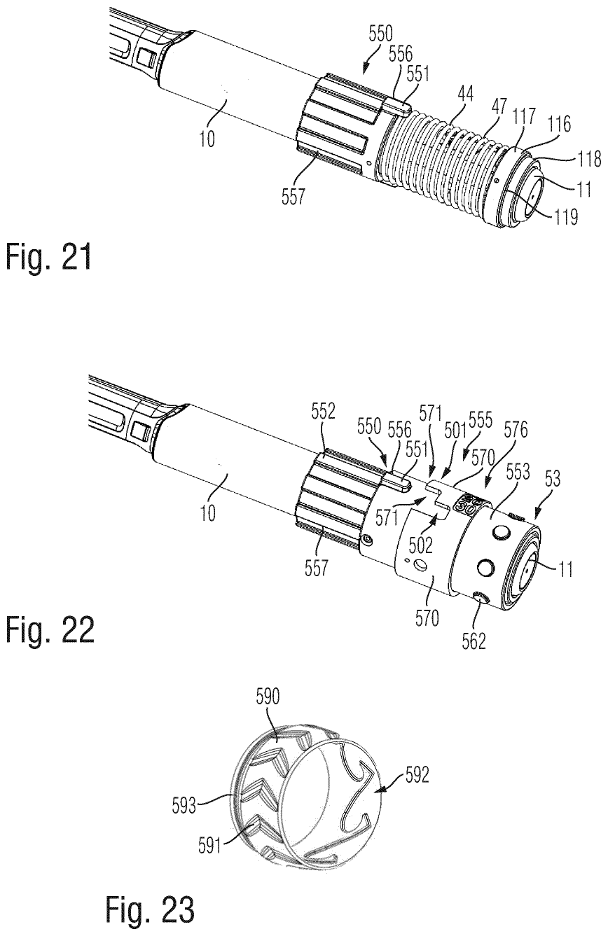

As illustrated in FIG. 21 in connection with FIG. 28 or FIG. 29 there is also provided a spring 44 implemented as a torsion spring 47. The spring 44 has a first end 45 permanently connected to the dose tracker 550, in particular to the distal part 552. Since the dose tracker 550 is rotationally fixed to the outer housing 100 the first end 45 of the spring 44 is effectively connected to the outer housing 100 and hence to the housing 10. In other words the first end 45 of the spring 44 is indirectly connected or coupled to the housing 10.

The opposite second end 46 of the spring 44 is connected to the dose dial 12 or to a separate sleeve-shaped fastener 116 as for instance illustrated in FIG. 21. The fastener 116 is annular shaped and comprises a ring structure. The fastener 116 is permanently locked or attached to the dose dial 12 provided at the proximal end of the injection device. The fastener 116 may be adhesively attached to the dose dial 12. The second end 46 of the spring 44 is connected to the fastener 116 in a torque prove way. Liberating the dose setting mechanism, e.g. by actuating the release member 590 enables a rotation of the number sleeve 80 and the rotation of the dose dial 12 will stop. As illustrated further the fastener 116 comprises a rim 117 and a recessed portion 118 on the outside surface of the fastener 116. The rim 117 extends into the recessed portion 118 via a radial step 119 or shoulder.

As illustrated in FIG. 28 the dose tracker 550, in particular the proximal part 553 comprises a radially inwardly extending ledge or rim 558 that is in axial abutment with the step 119. Insofar the rim 117 is in axial abutment with the rim 558. As the spring 44 induces a dose incrementing rotation of the fastener 116 and hence of the dose dial 12 the number sleeve 80 starts to rotate relative to the inner housing 10. Due to the threaded engagement between the insert 62 and the number sleeve 80 the number sleeve 80 and hence the dose dial 12 as well as the fastener 116 become subject to a proximally directed displacement relative to the outer housing 100. This proximal displacement of the fastener 116 is equally transferred to the dose tracker 550 due to the mutual axial abutment and engagement between the rim 117 and the rim 558.

A spring driven rotation of the number sleeve 80 therefore transfers to a longitudinal sliding and proximal displacement of the dose tracker 550 until the tracking stop feature 551 thereof engages with the preselector stop feature 571. As illustrated in FIGS. 28 and 29 there is provided a separate trigger 511 that covers the trigger 11 of the injection device 1. The trigger 511 is provided and configured to cover the trigger 11. The trigger 511 comprises a larger cross-section compared to the cross-section of the trigger 11. The trigger 511 may be adhesively attached to the trigger 11. The trigger 511 is configured to cover a proximal end of the outer housing 100.

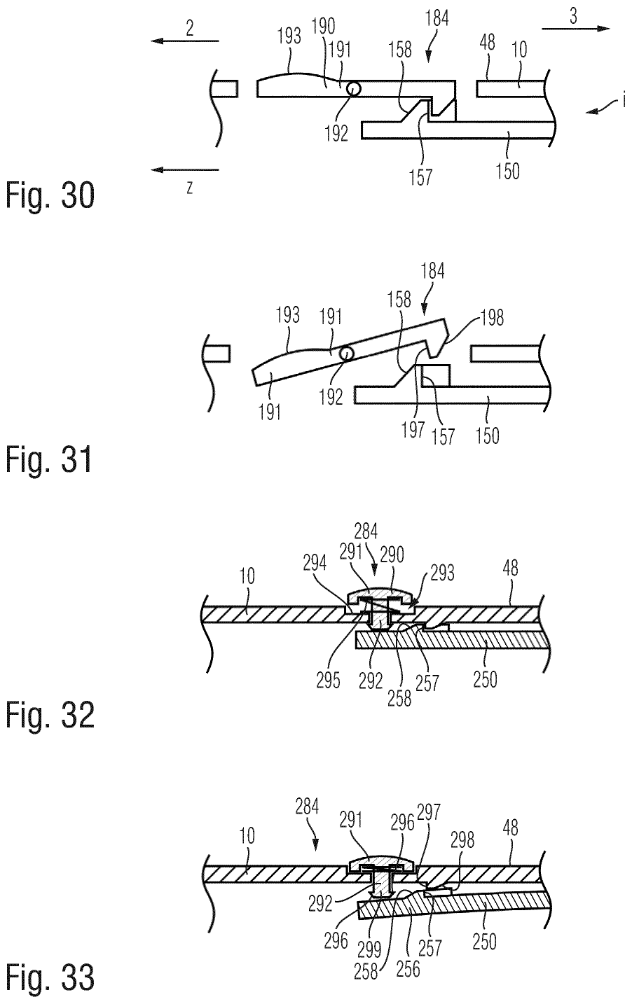

In FIGS. 30 and 31 a more detailed exemplary implementation of an interlock 184 and a release member 190 is illustrated. Here, the interlock 184 comprises a first locking feature provided on the dose tracker 150 and further comprises a second locking feature provided on the release member 190. The first locking feature is presently implemented as a catch 157 protruding radially outwardly from the dose tracker 150. The catch 157 may be integrally formed with the dose tracker 150. The release member 190 comprises a correspondingly shaped catch 197 protruding radially inwardly from the release member 190. The catch 197 may be also integrally formed with the release member 190.

The release member 190 is configured as a pivotable lever 191. The lever 191 is pivotally supported on a pivot axis 192. The pivot axis extends in tangential or circumferential direction with regard to the overall geometry of the housing 10. The lever 191 may flush with the outside surface of the sidewall of the housing 10 in the initial configuration i as shown in FIG. 30.

The lever 191 comprises the catch 197 and a depressible end portion at an opposite end. The depressible end portion and the catch 197 are provided on opposite ends of the lever 191. By depressing the depressible end radially inwardly the opposite end and hence the catch 197 is raised or lifted radially outwardly thus disengaging from the catch 157 of the dose tracker 150 as illustrated in FIG. 31. The release member 190 may be further provided with a return spring, presently not illustrated. The return spring may be arranged at the pivot axis 192 in order to return the release member 190 into the initial configuration as shown in FIG. 48, in which the catch 197 of the release member 190 is in axial abutment and in engagement with the correspondingly shaped catch 157 of the dose tracker 150.

The catch 157 comprises an axial abutment face facing in proximal direction. The catch 197 comprises a correspondingly shaped axial abutment surface facing in distal direction. In the initial configuration as illustrated in FIG. 30 the two abutment faces are in axial abutment thus inhibiting a proximally directed displacement of the dose tracker 150.

In one embodiment the release member 190 may comprise a radially outwardly bulged portion 193 that is configured to become depressed by the user of the device. The radially raised or bulged portion 193 slightly protrudes from the outside surface of the sidewall of the housing 10. Insofar it provides a haptic feedback to the user that this respective bulged portion 193 is configured for a radially inwardly directed depression. Once the user depresses the bulged portion 193 the oppositely located end section of the lever 191 is raised so that the mutually corresponding abutment faces 157, 197 get out of engagement. As the dose tracker 150 and hence the interlock 184 is liberated, the dose tracker 150 is free to rotate or to move proximally in longitudinal direction under the effect of the spring 44 as described above, e.g. in connection with FIGS. 3 to 6.

The catch 157 further comprises a beveled section 158. The catch 197 also comprises a correspondingly shaped beveled section 198. The beveled section 158 of the dose tracker 150 faces in distal direction 2 whereas the beveled section 198 of the release member 190 faces in proximal direction 3. During dose delivery the dose tracker 150 is subject to a distally directed displacement, hence to the left in FIGS. 30 and 31. As the catch 157 approaches the initial configuration or initial axial position as indicated in FIG. 30, the beveled section 158 slides along the beveled section 198. Such a sliding motion is accompanied by the release member 190 becoming lifted radially outwardly so that the outermost and inner most radial tips of the catches 157, 197 mutually pass by until the axial abutment faces 157, 197 return into an engagement configuration as shown in FIG. 30.

If the release member 190 or its lever 191 biased by a spring, the catch 197 is raised or lifted radially outwardly against the action of the respective spring. As soon as the abutment faces 197, 157 get in alignment the lever 191 snaps into the initial configuration i as illustrated in FIG. 30 under the action of the spring.

In FIGS. 32 and 33 a further conceivable implementation of an interlock 284 and a release member 290 is illustrated. Here, the dose tracker 250 comprises an elastic portion 256. The elastic portion 256 may axially protrude from the dose tracker 250. Alternatively, it may be integrated into the sidewall of the dose tracker 250. It may be separated from a sidewall of the dose tracker along a u-shaped slit. Here, the dose tracker 250 comprises a catch 257 correspondingly shaped with a catch 297 provided at an inside facing portion of the sidewall of the housing 10. The catch 257 comprises an axial abutment face as described above that faces in proximal direction 3. The correspondingly shaped catch 297 of the housing 10 comprises a distally facing abutment face to engage with or to abut with the abutment face 257.

The catch 257 and the catch 297 both comprise a beveled section 258, 298 that enable and induce a slight radially inwardly directed elastic deformation of the elastic portion 256 as the dose tracker 250 returns into the initial configuration as illustrated in FIG. 32.

The interlock 284 is formed by the mutually corresponding catches 257, 297 of the dose tracker 250 and the housing 10. In order to release the interlock 284 there is provided a release member 290 in form of a depressible button 291. The release member 290 comprises a somewhat planar-shaped or slightly bulged button 291 integrally formed with a longitudinally extending stem 292. The stem 292 extends radially inwardly and intersect a recess or through opening in the sidewall of the housing 10. The button 291 slightly protrudes from the outside surface of the sidewall of the housing 10. It is radially displaceably supported on the housing 10 against the action of a spring 295. The spring 295 is located in a recess 293 on the outside surface of the sidewall. The recess 293 comprises a bottom 294 that is recessed compared to the outside surface of the sidewall. The bottom 294 provides a support for the spring 295. An opposite end of the spring 295 is in abutment with an underside of the button 291.

An inner free end 299 of the stem 292 protrudes radially inwardly from an inside surface of the sidewall. The free end 299 is provided with lateral protrusions 296 that are separated by distance that is larger than the inner diameter of the recess of the sidewall through which the stem 292 extends. In this way, the stem 292 and the entire button 291 is hindered from getting pushed out of the housing 10 under the action of the spring 295.

In an initial configuration as illustrated in FIG. 32 the free end 299 of the stem 292 axially overlaps with the elastic portion 256 of the dose tracker 250. By depressing the release member 290 and hence the button 291 radially inwardly, the stem 292 advances downward in the illustration of FIG. 32 and FIG. 33. Since the free end 299 is in abutment with an outside surface portion of the elastic portion 256 such a depression leads to a local and radially inwardly directed deformation of the elastic portion 256. The elastic deformation is large enough to bring the catches 297, 298 out of engagement so as to liberate a proximally directed displacement of the dose tracker 250.

The examples of FIGS. 30 to 33 are only exemplary for an interlock and a release member 190, 290 and can be generally implemented with any of the examples as illustrated in FIG. 1 to FIG. 29.

List of reference numbers

| | |
|---|---|
| 1 | injection device |
| 2 | distal direction |
| 3 | proximal direction |
| 4 | dose incrementing direction |
| 5 | dose decrementing direction |
| 6 | cartridge |
| 7 | bung |
| 8 | drive mechanism |
| 9 | dose setting mechanism |
| 10 | housing |
| 11 | trigger |
| 12 | dose dial |
| 13 | dosage window |
| 14 | cartridge holder |
| 15 | injection needle |
| 16 | inner needle cap |
| 17 | outer needle cap |
| 18 | protective cap |
| 20 | piston rod |
| 21 | bearing |
| 22 | first thread |
| 23 | pressure foot |
| 24 | second thread |
| 25 | barrel |
| 26 | seal |
| 28 | threaded socket |
| 30 | drive sleeve |
| 31 | threaded section |
| 32 | flange |
| 33 | flange |
| 35 | last dose limiter |
| 36 | shoulder |
| 40 | spring |
| 41 | distal end |
| 42 | proximal end |
| 43 | preselection indication |
| 44 | spring |
| 45 | first end |
| 46 | second end |
| 47 | torsion spring |
| 48 | sidewall |
| 50 | dose tracker |
| 51 | tracking stop feature |
| 52 | zero dose positional state |
| 53 | proximal end |
| 54 | maximum dose positional state |
| 55 | tracking sleeve |
| 56 | protrusion |
| 60 | clutch |
| 62 | insert |
| 63 | protrusion |
| 64 | stem |
| 65 | spring |
| 66 | clutch |
| 67 | recess |
| 68 | sidewall |
| 70 | preselector |
| 71 | preselector stop feature |
| 72 | preselection positional state |
| 74 | preselection positional state |
| 75 | preselection indication |
| 80 | number sleeve |
| 81 | groove |
| 90 | release member |
| 100 | outer housing |
| 101 | groove |
| 102 | groove |
| 103 | groove |
| 104 | connecting groove |
| 107 | groove |
| 113 | preselection window |
| 114 | fastener |
| 116 | fastener |
| 117 | rim |
| 118 | recessed portion |
| 119 | step |
| 150 | dose tracker |
| 151 | tracking stop feature |
| 155 | tracking sleeve |
| 157 | catch |
| 158 | catch |
| 170 | preselector |
| 171 | preselector stop feature |
| 176 | protrusion |
| 184 | interlock |
| 190 | release member |
| 191 | lever |
| 192 | pivot axis |
| 193 | bulged portion |
| 197 | catch |
| 198 | catch |
| 250 | dose tracker |
| 251 | tracking stop feature |
| 255 | tracking sleeve |
| 256 | protrusion |
| 257 | catch |
| 258 | catch |
| 270 | preselector |
| 271 | preselector stop feature |
| 272 | preselector stop feature |
| 273 | preselector stop feature |
| 276 | protrusion |
| 284 | interlock |
| 290 | release member |
| 291 | button |
| 292 | stem |
| 293 | recess |
| 294 | bottom |
| 295 | spring |
| 296 | protrusion |
| 297 | catch |
| 298 | catch |
| 299 | free end |
| 350 | dose tracker |
| 351 | tracking stop feature |
| 355 | tracking sleeve |
| 356 | protrusion |
| 370 | preselector |
| 371 | preselector stop feature |
| 372 | preselector stop feature |
| 373 | preselector stop feature |
| 376 | protrusion |
| 450 | dose tracker |
| 451 | tracking stop feature |
| 455 | tracking sleeve |
| 470 | preselector |
| 471 | preselector stop feature |
| 476 | protrusion |
| 501 | recess |
| 502 | recess |
| 550 | dose tracker |
| 551 | tracking stop feature |
| 552 | distal part |
| 553 | proximal part |
| 555 | tracking sleeve |
| 556 | protrusion |
| 557 | rib |
| 558 | rim |
| 562 | protrusion |
| 570 | preselector |
| 571 | preselector stop feature |
| 572 | proximal end |
| 575 | locking feature |
| 576 | preselection indication |
| 590 | release member |
| 591 | annular ring |
| 592 | catch |
| 593 | groove |
| 594 | beveled section |
| 595 | curved section |
| 596 | undercut section |

1-15. (canceled)

16. An injection device for setting and injecting a dose of a medicament, the injection device comprising:
an elongated housing extending along a longitudinal axis and having a distal end and a proximal end,
a dose tracker being at least one of translationally or rotationally displaceable relative to the elongated housing and being displaceable between a zero dose positional state and a maximum dose positional state relative to the elongated housing for setting of the dose, wherein a positional state of the dose tracker relative to the elongated housing is indicative of a size of the dose,
wherein one of the elongated housing and the dose tracker comprises at least one tracking stop feature, and
a preselector displaceable relative to the elongated housing between at least two preselection positional states thereby defining the maximum dose positional state of the dose tracker, wherein the preselector comprises at least one preselector stop feature configured to mechanically engage with the at least one tracking stop feature to block a displacement of the dose tracker beyond the maximum dose positional state.

17. The injection device according to claim 16, wherein at least a proximal end of the dose tracker protrudes proximally from the proximal end of the elongated housing when in the maximum dose positional state, and wherein a longitudinal distance between the zero dose positional state and the maximum dose positional state correlates with the size of the dose.

18. The injection device according to claim 16, wherein the preselector is lockable relative to the elongated housing in any of the at least two preselection positional states.

19. The injection device according to claim 16, further comprising a release member connected to the dose tracker and selectively engageable to the elongated housing in order to lock the dose tracker to the elongated housing when in the zero dose positional state.

20. The injection device according to claim 16, further comprising a release member connected to the elongated housing and selectively engageable to the dose tracker in order to lock the dose tracker to the elongated housing when in the zero dose positional state.

21. The injection device according to claim 20, wherein the release member comprises an annular ring rotationally supported at the proximal end of the elongated housing, wherein an inside surface of the annular ring comprises at least one catch to engage with a protrusion on an outside surface of the dose tracker.

22. The injection device according to claim 20, wherein the release member comprises an annular ring rotationally supported at the proximal end of the elongated housing, wherein an outside surface of the dose tracker comprises at least one catch to engage with a protrusion on an inside surface of the annular ring.

23. The injection device according to claim 16, further comprising a spring having a first end operably connected to the elongated housing and having a second end operably connected to the dose tracker for displacing the dose tracker from the zero dose positional state to the maximum dose positional state.

24. The injection device according to claim 23, wherein the spring comprises a cylindrically shaped torsion spring and wherein the spring encloses at least a portion of the dose tracker or wherein the spring is arranged inside a hollow portion of the dose tracker.

25. The injection device according to claim 16, wherein the dose tracker comprises a tracking sleeve threadedly engaged with the elongated housing.

26. The injection device according to claim 16, wherein the at least one tracking stop feature comprises a radial protrusion protruding from a sidewall of the dose tracker.

27. The injection device according to claim 26, wherein the preselector stop feature comprises at least a first groove and a second groove configured to slidably receive the radial protrusion of the at least one tracking stop feature.

28. The injection device according to claim 27, wherein the first groove extends parallel to the second groove, wherein the second groove is longer than the first groove, wherein the first groove and the second groove merge into a connecting groove, and wherein the connecting groove extends along a direction along which the preselector is displaceable between a first preselection positional state and a second preselection positional state of the at least two preselection positional states.

29. The injection device according to claim 28, wherein the first and the second groove each comprise an end facing away from the connecting groove that defines the maximum dose positional state.

30. The injection device according to claim 16, wherein the preselector stop feature comprises a radial protrusion protruding from a sidewall of the preselector.

31. The injection device according to claim 30, wherein the at least one tracking stop feature comprises at least a first groove and a second groove configured to slidably receive the radial protrusion of the preselector stop feature.

32. The injection device according to claim 31, wherein the first groove extends parallel to the second groove, wherein the second groove is longer than the first groove, and wherein the first groove and the second groove merge into a connecting groove, wherein the connecting groove extends along a direction along which the preselector is displaceable between a first preselection positional state and a second preselection positional state of the at least two preselection positional states.

33. The injection device according to claim 16, wherein the preselector is rotationally supported on the elongated housing or wherein the preselector is displaceable relative to the elongated housing along a tangential or circumferential direction.

34. The injection device according to claim 16, further comprising a trigger and a piston rod, wherein the trigger is arranged at a proximal end of the dose tracker, and wherein the trigger is depressible in a distal direction to induce a distally directed motion of the piston rod.

35. The injection device according to claim 16, further comprising a cartridge wherein the cartridge comprises a barrel filled with the medicament and sealed by a bung that is axially displaceable relative to the barrel by means of a piston rod.
